(19) United States
(12) Patent Application Publication
(10) Pub. No.: US 2025/0345104 A1
(43) Pub. Date: Nov. 13, 2025

(54) METATARSOPHALANGEAL JOINT PREPARATION AND METATARSAL REALIGNMENT FOR FUSION

(71) Applicant: Treace Medical Concepts, Inc., Ponte Vedra, FL (US)

(72) Inventors: William T. DeCarbo, Mars, PA (US); Paul Dayton, Ankeny, IA (US); W. Bret Smith, Lexington, SC (US); Robert D. Santrock, Morgantown, WV (US); Daniel J. Hatch, Greeley, CO (US); Jody McAleer, Jefferson City, MO (US)

(21) Appl. No.: 19/275,797

(22) Filed: Jul. 21, 2025

Related U.S. Application Data
(63) Continuation of application No. 18/297,712, filed on Apr. 10, 2023, now Pat. No. 12,364,522.
Application No. 18/297,712 is a continuation of application No. 17/163,120, filed on Jan. 29, 2021, now Pat. No. 11,622,797.
(60) Provisional application No. 62/968,244, filed on Jan. 31, 2020.

(51) Int. Cl. A61B 17/72; A61B 17/00; A61B 17/68

(52) U.S. Cl. CPC A61B 17/7291; A61B 2017/00407; A61B 2017/681

(57) ABSTRACT

A method of preparing a metatarsophalangeal joint for fusion may involve surgically accessing the metatarsophalangeal joint and separating the metatarsal from the opposed proximal phalanx at the joint. The technique may involve preparing an end of the metatarsal and preparing an end of the opposed proximal phalanx for fusion. With or without the aid of a bone positioner, the metatarsal may be moved in one or more planes, such as the frontal and transverse planes. The proximal phalanx may also be moved in one or more planes, for example relative to the moved metatarsal. Subsequently, a bone fixation device can be applied across or through the metatarsophalangeal joint separating the metatarsal from the opposed proximal phalanx.

### RELATED MATTERS

This application is a continuation of U.S. patent application Ser. No. 18/297,712, filed Apr. 10, 2023, which is a continuation of U.S. patent application Ser. No. 17/163,120, filed Jan. 29, 2021, now U.S. Pat. No. 11,622,797, issued Apr. 11, 2023, which claims the benefit of U.S. Provisional Patent Application No. 62/968,244, filed Jan. 31, 2020. The entire contents of each of these applications are incorporated herein by reference.

### TECHNICAL FIELD

This disclosure relates to devices and techniques for metatarsophalangeal joint preparation and metatarsal realignment.

### BACKGROUND

The human foot includes the five toes (which are also known as the “phalanges”) and their connecting long bones (or “metatarsals”). The joint between a metatarsal and a phalange is called the metatarsophalangeal (“MTP”) joint. Several small bones together comprise a phalanx or toe. Four of the five toes have three phalanx bones respectively connected by two joints. The big toe (or “hallux”) has two phalanx bones distal and proximal with a joint in between called the interphalangeal joint. The big toe articulates with the head of the first metatarsal at the first metatarsophalangeal joint (the “MTP” joint) and there are two tiny, round bones called sesamoids on the plantar side of the metatarsal head. The phalanges are connected to the metatarsals at the ball of the foot. The two sesamoid bones are located underneath the first metatarsal bone and assist in flexion of the big toe at the first MTP joint.

Hallux rigidus is a degenerative disease of the first metatarsophalangeal joint. It is the most common form of arthritis in the foot, reportedly affecting 1 in 40 people over the age of 50. The first MTP joint plays an important functional role during the gait cycle as it carries a significant amount of an individual's body weight with each step. Osteophyte formation and degeneration of the cartilage occurs dorsally in early stages of the disease and progresses to involve the entire first MTP joint. Consequently, individuals with hallux rigidus experience joint pain and decreased range of motion in the sagittal plane. This leads to altered gait mechanics and significant reduction in activity and quality of life for patients.

### SUMMARY

In general, this disclosure is directed to devices and techniques for preparing the metatarsophalangeal (“MTP”) joint for fusion and realigning the metatarsal and phalanx separated by the joint. In some implementations, a clinician surgically accesses the MTP joint and separates the metatarsal from the opposed phalanx for subsequent bone preparation and realignment. The clinician may make a longitudinal incision, e.g., just medial to the extensor hallucis longus tendon. With the MTP joint exposed, the clinician may separate the end face of the metatarsal from the end face of the opposed phalanx to provide working space. The clinician may subsequently prepare the end face of the metatarsal and the end face of the opposed phalanx for fusion. Example preparation steps may include reaming, cutting, rongeuring, curetting, burring, fenstrating and/or other similar techniques for exposing subchondral bone and/or establishing bleeding bone faces to promote fusion following rejoining of the metatarsal and phalanx.

Either before or after preparation of one or both end faces, the metatarsal is realigned within one or more planes in three-dimensional space. In one example, the clinician engages a bone positioner with the metatarsal and a bone other than the metatarsal. The bone positioner can then be actuated to move the metatarsal in one or more planes for realignment. The use of the bone positioner can provide an instrumented approach to metatarsal realignment that allows for repeatable, consistent clinical outcomes patient-to-patient and clinician-to-clinician. In other examples, however, the clinician may realign the metatarsal manually without the aid of an instrumented bone positioner bridging between the metatarsal and another anchoring bone. The clinician may realign the metatarsal in one or more planes at a time, e.g., including the frontal plane.

Independent of the specific technique the clinician uses to realign the metatarsal, the clinician may also realign the proximal phalanx relative to the metatarsal. As one realignment, the clinician may adjust the orientation of the phalanx in the sagittal plane to help set a desired amount of dorsiflexion. As another example realignment, the clinician may rotate the phalanx within the frontal plane to help provide anatomically accurate positioning of the plantar side of the phalanx in the frontal plane.

To help guide accurate realignment and visualization of the proximal phalanx, the clinician may provisionally fixate the phalanx to an adjacent bone during and/or after realignment. For example, the clinician may insert a pin (e.g., a Kirschner wire, which is also referred to as a K wire) through the distal end of the phalanges and advance the pin proximally toward the metatarsal.

In the case of the first metatarsal, for instance, the clinician can insert the pin through the distal phalanx followed by the proximal phalanx and then lodge the distal portion of the pin in the first metatarsal. The pin can project distally out of the distal phalanx, providing a visible axis of rotation for realigning the proximal phalanx in the frontal plane. When so implemented, the clinician may first set the orientation of the phalanx in the sagittal and/or transverse plane and then pin the phalanx to the metatarsal in that orientation. The clinician can then set the orientation of the phalanx in the frontal plane, e.g., by rotating the phalanx in the frontal plane about the pin. Once the orientation of the phalanx is set in the frontal plane, provisional and/or permanent fixation can be used to hold the moved position of the phalanx relative to the metatarsal for subsequent fusion.

According to some example implementations of the devices and techniques described herein, the metatarsal and phalanx separated by the MTP joint can be independently realigned relative to each other. For example, the position of the metatarsal in three-dimensional space may first be adjusted to a desired moved position and provisionally held in that moved position during subsequent realignment of the phalanx. The position of the phalanx can then be adjusted to a desired moved position in three-dimensional space (e.g., relative to the metatarsal that has already been realigned). With the metatarsal and proximal phalanx each independently realigned relative to each other, one or more fixation devices can be applied across and/or through the MTP joint to promote subsequent fusion of the bones. For example, one or more plates, screws, pins, and/or the like can be applied to permanently fixate the metatarsal to the phalanx and promote fusion of the bones at the MTP joint.

Through the stepwise adjustment of the orientation of the metatarsal and opposed phalanx, the position of each bone can be adjusted from an anatomically misaligned position toward a position of anatomical alignment.

In one example, a method is described that includes preparing a metatarsophalangeal joint for fusion. The method includes surgically accessing a metatarsophalangeal joint separating a metatarsal from an opposed proximal phalanx and preparing an end of the metatarsal and preparing an end of the opposed proximal phalanx. The method also includes moving the metatarsal in at least two planes to establish a moved position of the metatarsal and applying at least one bone fixation device across or through the metatarsophalangeal joint separating a metatarsal from an opposed proximal phalanx.

In another example, a method of preparing a metatarsophalangeal joint for fusion is described. The method includes surgically accessing a metatarsophalangeal joint separating a metatarsal from an opposed proximal phalanx, preparing an end of the metatarsal and preparing an end of the opposed proximal phalanx, and engaging a bone positioning guide with the metatarsal and a bone other than the metatarsal. The method also includes actuating the bone positioning guide and thereby moving the metatarsal in at least one plane to establish a moved position of the metatarsal and applying at least one bone fixation device across or through the metatarsophalangeal joint separating a metatarsal from an opposed proximal phalanx.

The details of one or more examples are set forth in the accompanying drawings and the description below. Other features, objects, and advantages will be apparent from the description and drawings, and from the claims.

### BRIEF DESCRIPTION OF DRAWINGS

FIGS. 1A and 1B are front views of a foot showing a normal first metatarsal position and an example frontal plane rotational misalignment position, respectively.

FIGS. 2A and 2B are top views of a foot showing a normal first metatarsal position and an example transverse plane misalignment position, respectively.

FIGS. 3A and 3B are side views of a foot showing a normal first metatarsal position and an example sagittal plane misalignment position, respectively.

FIG. 4 is a flow diagram illustrating an example technique for preparing the MTP joint for fusion and realigning the metatarsal and proximal phalanx separated by the joint.

FIG. 5 is a perspective view of a foot showing example surgical access to the MTP joint.

FIG. 6 is a side view of a first metatarsal and sesamoid complex showing the position of the sesamoid bones when in proper anatomical alignment.

FIG. 7 is a side view of the first metatarsal and sesamoid complex from FIG. 6 showing an example anatomical misalignment of the sesamoid bones.

FIG. 8 is a perspective image of a foot illustrating an example preparation step performed on first metatarsal.

FIG. 9 is a perspective illustration of an example bone positioning device that can be used to move a metatarsal.

FIG. 10 is a perspective view of a foot showing the bone positioning device of FIG. 9 attached to the foot.

FIG. 11 is a perspective view of a foot showing an example pin inserted into the hallux of the foot and into the first metatarsal.

FIG. 12 is a fluoroscopic image of the foot of FIG. 11 taken in the dorsal to plantar direction.

FIG. 13 is a perspective view of a foot showing a compression pin inserted through the proximal phalanx and into the first metatarsal along with an example bone plate.

FIG. 14 is a perspective view of a foot showing example bi-planar bone plates.

FIGS. 15A-15D are illustrations of example surgical steps that may be performed to engage a compressor with a metatarsal and opposed proximal phalanx to facilitate compression.

FIGS. 16 and 17 are pre-and post-operative fluoroscopic images of a foot showing example bone realignment according to the techniques of the present disclosure.

### DETAILED DESCRIPTION

In general, the present disclosure is directed to devices and techniques for preparing the metatarsophalangeal (“MTP”) joint for fusion and realigning the metatarsal and phalanx separated by the joint. While a technique according to the disclosure can be performed on any MTP joint where a metatarsal is joined to an opposing proximal phalanx, in some implementations, the technique is performed on the first MTP joint where the first metatarsal joins the first proximal phalanx. During the procedure, a metatarsal may be separated from an opposing proximal phalanx at the MTP joint and both the metatarsal and opposing phalanx repositioned within one or more planes. After suitably repositioning the metatarsal and opposed proximal phalanx, the bone portions may be fixed to each using one or more fixation devices crossing the MTP joint. The end faces of the metatarsal and opposed proximal phalanx can be prepared prior to fixation, e.g., to promote realignment and/or subsequent fusion of the bones to each other.

Preparation and fusion of a metatarsal and phalanx may be performed according to the disclosure for a variety of clinical reasons and indications. Preparation and fusion of a metatarsal and proximal phalanx at the MTP joint may be performed to treat hallux rigidus, hallux valgus, and/or other bone and/or joint conditions.

Hallux rigidus is characterized as a degenerative arthritis of the MTP joint, particularly the first MTP joint. The cause of hallux rigidus is often unclear. While arthritis can be caused by traumatic or iatrogenic injuries that directly cause damage to the articular cartilage of the MTP joint, most commonly the aetiology of hallux rigidus is idiopathic. Patients may have family history and/or bilateral involvement leading to hallux rigidus. As hallux rigidus progresses, the normal coupling of the center of rotation of the proximal phalanx and metatarsal head may be disrupted, leading to eccentric gliding of proximal phalanx on the metatarsal head. Osteophytes may form preferentially on the dorsal surface. Further, while a normal first MTP motion may have approximately 75° of dorsiflexion and 35° of plantarflexion, patients with hallux rigidus may typically exhibit a decreased range of motion, such as dorsiflexion less than 60°, such as less than 40°, less than 30°, or even less than 20°.

Hallux valgus, also referred to as hallux abducto valgus, is a complex progressive condition that is characterized by lateral deviation (valgus, abduction) of the hallux and medial deviation of the first metatarsophalangeal joint. Hallux valgus typically results in a progressive increase in the hallux abductus angle, the angle between the long axes of the first metatarsal and proximal phalanx in the transverse plane. An increase in the hallux abductus angle may tend to laterally displace the plantar aponeurosis and tendons of the intrinsic and extrinsic muscles that cross over the first metatarsophalangeal joint from the metatarsal to the hallux. Consequently, the sesamoid bones may also be displaced, e.g., laterally relative to the first metatarsophalangeal joint, resulting in subluxation of the joints between the sesamoid bones and the head of the first metatarsal. This can increase the pressure between the medial sesamoid and the crista of the first metatarsal head.

While the techniques and devices are described herein particularly in connection with the first metatarsal and first proximal phalanx of the foot, the techniques and devices may be used on other adjacent bones separated by a joint in the hand or foot. For example, the techniques and devices may be performed on a different metatarsal (e.g., second, third, fourth, or fifth metatarsal) and its opposed proximal phalanx.

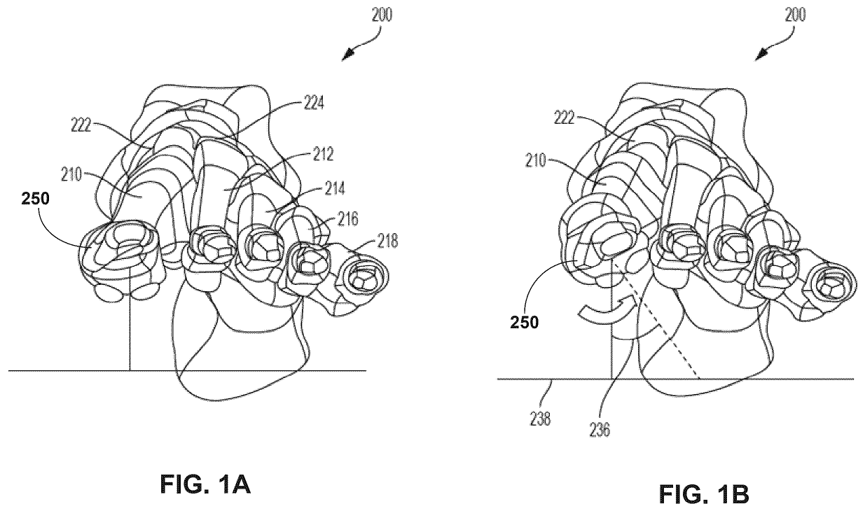

To further understand example techniques of the disclosure, the anatomy of the foot will first be described with respect to FIGS. 1-3 along with example misalignments that may occur and be corrected according to the present disclosure. As noted, a bone misalignment and/or MTP joint pain may be caused by hallux valgus (bunion), hallux rigidus, a natural growth deformity, and/or other condition. The condition may present with a misalignment of one or more bones in the foot. Alternatively, the condition may present with evidence of arthritis at the MTP joint without visible misalignment of the bone forming the joint.

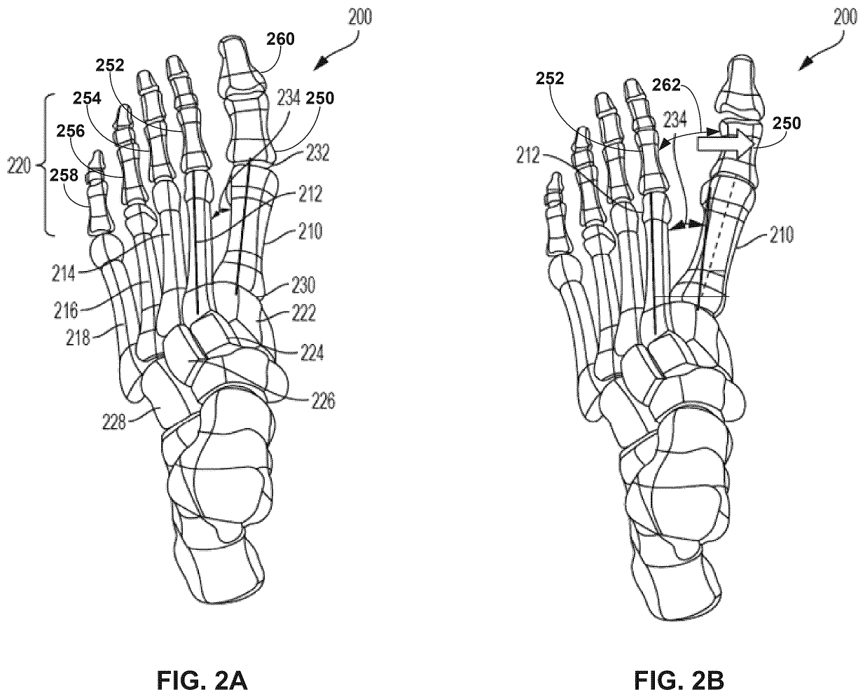

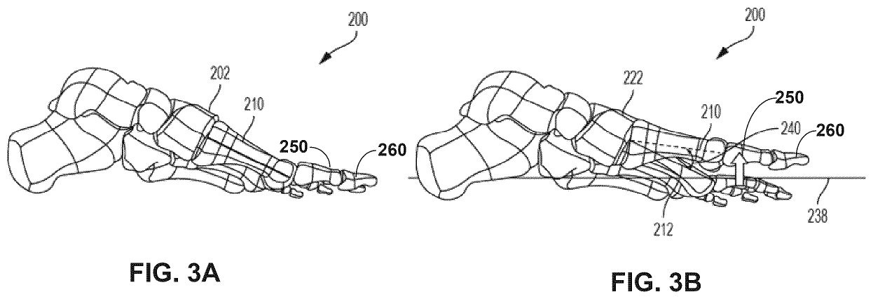

FIGS. 1A and 1B are front views of foot 200 showing a normal first metatarsal position and an example frontal plane rotational misalignment position, respectively. FIGS. 2A and 2B are top views of foot 200 showing a normal first metatarsal position and an example transverse plane misalignment position, respectively. FIGS. 3A and 3B are side views of foot 200 showing a normal first metatarsal position and an example sagittal plane misalignment position, respectively. While FIGS. 1B, 2B, and 3B show each respective planar misalignment in isolation, in practice, a metatarsal may be misaligned in any two of the three planes or even all three planes. Accordingly, it should be appreciated that the depiction of a single plane misalignment in each of FIGS. 1B, 2B, and 3B is for purposes of illustration and a metatarsal may be misaligned in multiple planes that is desirably corrected. Further, a bone condition treated according to the disclosure may not present any of the example misalignments described with respect to FIGS. 1B, 2B, and 3B, and it should be appreciated that the disclosure is not limited in this respect.

With reference to FIGS. 1A and 2A, foot 200 is composed of multiple bones including a first metatarsal 210, a second metatarsal 212, a third metatarsal 214, a fourth metatarsal 216, and a fifth metatarsal 218. The metatarsals are connected distally to phalanges 220 and, more particularly, each to a respective proximal phalanx. In particular, the first metatarsal 210 is connected distally to first proximal phalanx 250, the second metatarsal 212 is connected distally to second proximal phalanx 252, the third metatarsal 214 is connected distally to third proximal phalanx 254, the fourth metatarsal 216 is connected distally to fourth proximal phalanx 256, and the fifth metatarsal 218 is connected distally to fifth proximal phalanx 258. The joint 232 between a metatarsal and a corresponding opposed proximal phalanx is referred to as a metatarsophalangeal (“MTP”) joint. The first MTP joint is labeled as joint 232 in FIG. 2A, although second, third, fourth, and fifth MTP joints are also illustrated in series adjacent to the first MTP joint.

The first metatarsal 210 is connected proximally to a medial cuneiform 222, while the second metatarsal 212 is connected proximally to an intermediate cuneiform 224 and the third metatarsal is connected proximally to lateral cuneiform 226. The fourth and fifth metatarsals 216, 218 are connected proximally to the cuboid bone 228. The joint 230 between a metatarsal and respective cuneiform (e.g., first metatarsal 210 and medial cuneiform 222) is referred to as the tarsometatarsal (“TMT”) joint. The angle 234 between adjacent metatarsals (e.g., first metatarsal 210 and second metatarsal 212) is referred to as the intermetatarsal angle (“IMA”).

As noted, FIG. 1A is a frontal plane view of foot 200 showing a typical position for first metatarsal 210. The frontal plane, which is also known as the coronal plane, is generally considered any vertical plane that divides the body into anterior and posterior sections. On foot 200, the frontal plane is a plane that extends vertically and is perpendicular to an axis extending proximally to distally along the length of the foot. FIG. 1A shows first metatarsal 210 in a typical rotational position in the frontal plane. FIG. 1B shows first metatarsal 210 with a frontal plane rotational deformity characterized by a rotational angle 236 relative to ground, as indicated by line 238.

FIG. 2A is a top view of foot 200 showing a typical position of first metatarsal 210 in the transverse plane. The transverse plane, which is also known as the horizontal plane, axial plane, or transaxial plane, is considered any plane that divides the body into superior and inferior parts. On foot 200, the transverse plane is a plane that extends horizontally and is perpendicular to an axis extending dorsally to plantarly (top to bottom) across the foot. FIG. 2A shows first metatarsal 210 with a typical IMA 234 in the transverse plane. FIG. 2B shows first metatarsal 210 with a transverse plane rotational deformity characterized by a greater IMA caused by the distal end of first metatarsal 210 being pivoted medially relative to the second metatarsal 212.

FIG. 3A is a side view of foot 200 showing a typical position of first metatarsal 210 in the sagittal plane. The sagittal plane is a plane parallel to the sagittal suture which divides the body into right and left halves. On foot 200, the sagittal plane is a plane that extends vertically and intersects an axis extending proximally to distally along the length of the foot. FIG. 3A shows first metatarsal 210 with a typical rotational position in the sagittal plane. FIG. 3B shows first metatarsal 210 with a sagittal plane rotational deformity characterized by a rotational angle 240 relative to ground, as indicated by line 238.

Bone positioning techniques and instruments can be useful to correct a misalignment of one or more bones, such as the metatarsal and opposed proximal phalanx, and/or promote fusion of the metatarsal and proximal phalanx across the MTP joint. In some applications, the technique involves releasing the MTP joint and preparing the end faces of the metatarsal and proximal phalanx for realignment relative to each other and/or fusion. The metatarsal undergoing the procedure may be moved in at least two planes, such as all three planes, to provide a moved position for fusing with the proximal phalanx. The proximal phalanx undergoing the procedure may also be moved in at least one plane, such as two or all three planes, relative to the metatarsal and/or an adjacent proximal phalanx. Once the metatarsal and proximal phalanx are appropriately repositioned, the metatarsal and proximal phalanx can be fixated to hold and maintain their relative positions to each other, e.g., and to promote fusion between the bones.

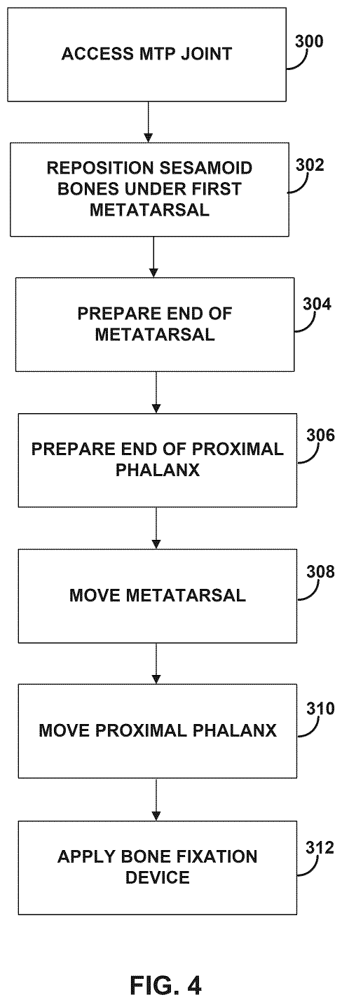

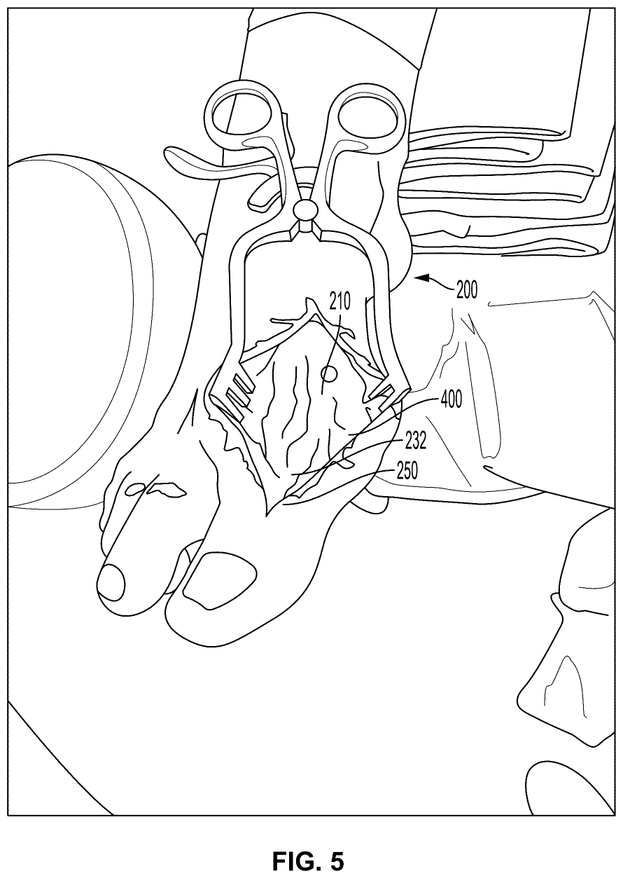

FIG. 4 is a flow diagram illustrating an example technique for preparing the MTP joint for fusion and realigning the metatarsal and proximal phalanx separated by the joint. The technique will be described with respect to first metatarsal 210 and first proximal phalanx 250, which are joined at the first MTP joint 232, although can be performed on other bones, as discussed above. For purposes of discussion, the technique of FIG. 4 will be discussed with respect to different example images of a procedure illustrated in FIGS. 5-16.

FIG. 5 is a perspective view of a foot 200 showing example surgical access to the MTP joint. With reference to FIGS. 4 and 5, the example technique involves surgically accessing metatarsophalangeal joint 232 separating first metatarsal 210 from its opposed proximal phalanx 250 (300). To surgically access the joint, the patient may be placed in a supine position on the operating room table and general anesthesia or Monitored Anesthesia Care (MAC) administered. Hemostasis can be obtained by applying thigh tourniquet or mid-calf tourniquet. An incision 400 (FIG. 5) can be made on a dorsal side of the foot 200, such as a dorsal-medial side of the foot. For example, incision 400 may be made just medial to the extensor hallucis longus tendon centered over the first MTP joint 232. In some applications, a full thickness sub-periosteal dissection is carried out exposing the first MTP joint 232.

With the MTP joint 232 exposed, first metatarsal 210 may be separated from first proximal phalanx 250 at the joint. For example, first proximal phalanx 250 may be pushed plantarly, resulting in the end face of the first proximal phalanx separating from the end face of first metatarsal 210 to provide access to both end faces for subsequent operation. If needed, a soft tissue release performed at MTP joint 232 to help separate and release the metatarsal from the phalanx. When present, exuberant bony exostosis can be removed from the head (e.g., ed face) of the first metatarsal 210 and the base (e.g., end face) of the proximal phalanx 250.

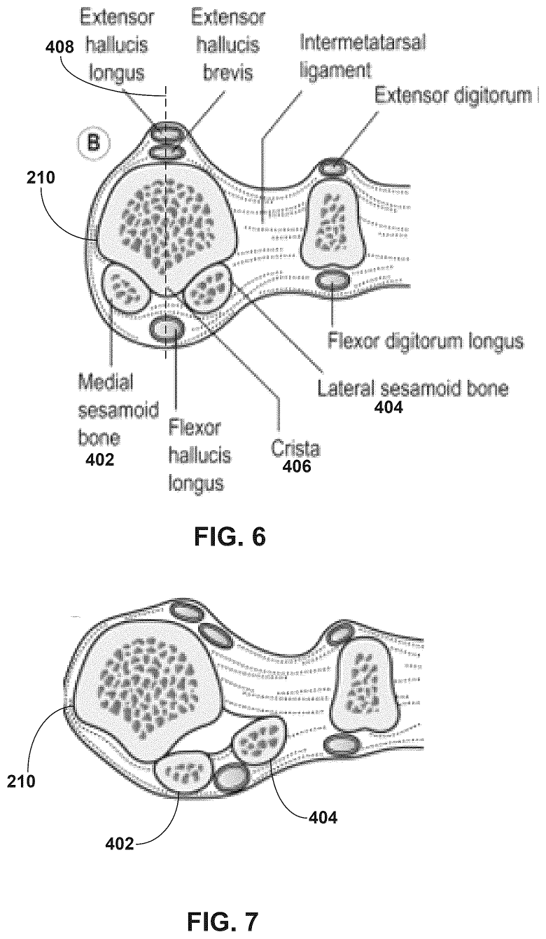

In some examples of the technique of FIG. 4, the sesamoid complex is repositioned under the first metatarsal 210 (302). FIG. 6 is a side view of a first metatarsal and sesamoid complex showing the position of the sesamoid bones when in proper anatomical alignment. With reference to FIG. 6, the sesamoid bones 402, 404 are two bones positioned under the first metatarsal 210 when the bones and metatarsal are in proper anatomical realignment. The medial and lateral aspects of the plantar surface of the head of the first metatarsal 210 normally form shallow parallel grooves on either side of the central sagittal ridge called the crista 406. The medial and lateral sesamoid bones articulate with the medial and lateral grooves, respectively. The sesamoid bones slide forward in their respective grooves during extension (windlass action) and backward during flexion (reverse windlass action) of the first MTP joint 232 when in proper alignment.

In the case of a misalignment, however, the sesamoid bones 402, 404 may be displaced (e.g., laterally) relative to the first MPT joint 232. FIG. 7 is a side view of the first metatarsal and sesamoid complex from FIG. 6 showing an example anatomical misalignment of the sesamoid bones. Displacement of the sesamoid bones 402, 404 may cause increased pressure between the medial sesamoid 402 and the crista 406 of the first metatarsal head 210 and decreased pressure between the lateral sesamoid 404 and the first metatarsal head.

In the technique of FIG. 4, the medial sesamoid bone 402 and the lateral sesamoid bone 404 may be repositioned on opposite sides of the sagittal plane 408. For example, the sesamoid bones 402, 404 may be positioned on opposite sides of the crista 406 within the respective parallel grooves formed by the head of the first metatarsal 210. To reposition the sesamoid bones, an instrument may be used to substantially fully release the capsular tissue at the MTP joint and for complete release of the sesamoids from the metatarsal. For example, a McGlamery elevator instrument may be used inserted (e.g., plantarly of the metatarsal) to release capsular tissue and to release the sesamoids, although a number of other instruments can be used instead of this particular device. The sesamoid bones 402, 404 may naturally realign upon being released from the first metatarsal 210, although manual manipulation or rotation (e.g., with or without the aid of a further instrument) may be used to assist realignment of the sesamoid bones.

Before or after the sesamoid bones are optionally repositioned in the technique of FIG. 4 (302), the end face of the first metatarsal 210 and/or the end face of the proximal phalanx 250 may be prepared (304, 306). Bone preparation can be useful, for instance, to facilitate contact between leading edges of metatarsal 210 and proximal phalanx 250. Preparation of the end faces may allow the two bones to be realigned relative to each other and/or to prepare the end faces to promote fusion between the end faces.

To prepare the end face of the first metatarsal 210 and/or the end face of the first proximal phalanx 250, a tissue removing instrument can be applied to the end face. Example tissue removing instruments that can be used include, but are not limited to, a saw, a rotary bur, a rongeur, a reamer, an osteotome, and the like. The tissue removing instrument can be applied to the end face of the bone being prepared to remove cartilage and/or bone. For example, the tissue removing instrument may be applied to the end face to remove cartilage (e.g., all cartilage) down to subchondral bone. Additionally or alternatively, the tissue removing instrument may be applied to cut, fenestrate, morselize, and/or otherwise reshape the end face of the bone and/or form a bleeding bone face to promote fusion. In instances where a cutting operation is performed to remove an end portion of first metatarsal 210 and/or first proximal phalanx 250, the cutting may be performed freehand or with the aid of cutting guide having a guide surface positionable over the portion of bone to be cut. When using a cut guide, a cutting instrument can be inserted against the guide surface (e.g., between a slot define between two guide surfaces) to guide the cutting instrument for bone removal.

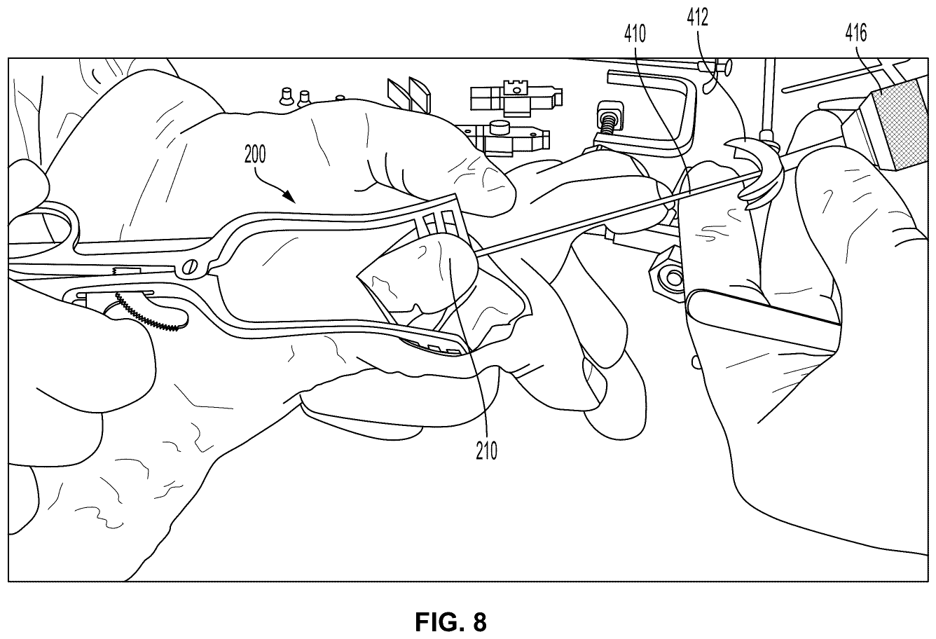

FIG. 8 is a perspective image of foot 200 illustrating an example preparation step performed on first metatarsal 210 (304). In the illustrated example, a guide pin 410 is inserted to project distally out of the end face of first metatarsal 210. A generally concave reamer 412 (e.g., a cone reamer) is advanced axially down the guide pin 412 and rotated using a rotary instrument 416 (e.g., drill). Reamer 412 (or other instrument, if a different surgical instrument is used) may denude the end face of the first metatarsal 210 of remaining cartilage down to the subchondral bone plate. In some examples, the end face of the first metatarsal 210 may be fenestrated in addition to or in lieu of one or more other end preparation steps, such as reaming the face with reamer 412.

The proximal phalanx 250 may be prepared using the same preparation technique or techniques performed on first metatarsal 210 or using a different preparation technique or techniques (306). In instances where the end face of the first metatarsal is prepared using a generally conically-shaped reamer 412, the end face of the first proximal phalanx 250 may be prepared using a corresponding generally convex reamer (e.g., a cup reamer). A guide pin may be inserted into the end face of the first proximal phalanx 250 and the convex reamer advanced down the guide pin and rotated using a rotary instrument. The reamer (or other instrument, if a different surgical instrument is used) may denude the end face of the proximal phalanx 250 of remaining cartilage down to the subchondral bone plate. In some examples, the end face of the proximal phalanx 250 may be fenestrated in addition to or in lieu of one or more other end preparation steps, such as reaming the face with the reamer.

In general, the specific order of the surgical steps performed in the technique of FIG. 4 may be varied without departing from the scope disclosure, and the example order illustrated in FIG. 4 it is merely for purposes of illustration. For example, the end face of the first metatarsal 210 may be prepared before or after the end face of the proximal phalanx 250 is prepared. Further, although FIG. 4 illustrates the end faces of the two bones as being prepared before moving either of the bones for realignment, one or both bones may be moved to a realigned position, as will be described below, before preparing an end face of one or both bones.

The technique of FIG. 4 involves moving the first metatarsal (e.g., relative to an adjacent metatarsal, such as a second metatarsal 212, and/or the first proximal phalanx 250) to establish a moved position of the metatarsal (308). In different examples, movement of the first metatarsal may be performed freehand (e.g., without the aid of a bone positioning device) and/or using instrumentation (e.g., a bone positioning device) to help facilitate repeatable repositioning outcomes.

When a bone positioning device is used, the bone positioning device may be any instrument that engages with the metatarsal being repositioned (e.g., first metatarsal 210) and a bone other than the metatarsal being repositioned. For example, the bone positioning device may engage with the metatarsal 210 on one side and another bone that acts as an anchor for the bone positioning device during actuation and corresponding movement of the metatarsal. The other bone used to anchor the bone positioning device may be another metatarsal (e.g., second metatarsal 212 or yet other metatarsal), a cuneiform such as medial cuneiform 222, or yet other anchoring bone. The bone positioning device may engage with the metatarsal being moved and the other bone through frictional contact without being fixedly coupled to the bone. Additionally or alternatively, a pin, screw, and/or other fixation element may be used to secure the bone positioning device to one or both bones.

In general, a bone positioning device may apply a force to a bone that causes the bone to move in at least one plane, such as the transverse plane and/or the frontal plane. In some examples, the force applied by the bone positioning device moves the metatarsal to which the force is applied in multiple planes, such as at least two planes or all three planes. For example, the bone positioning device may cause the first metatarsal 210 to be moved in the transverse plane to close the intermetatarsal angle and may also cause the metatarsal to rotate in the frontal plane.

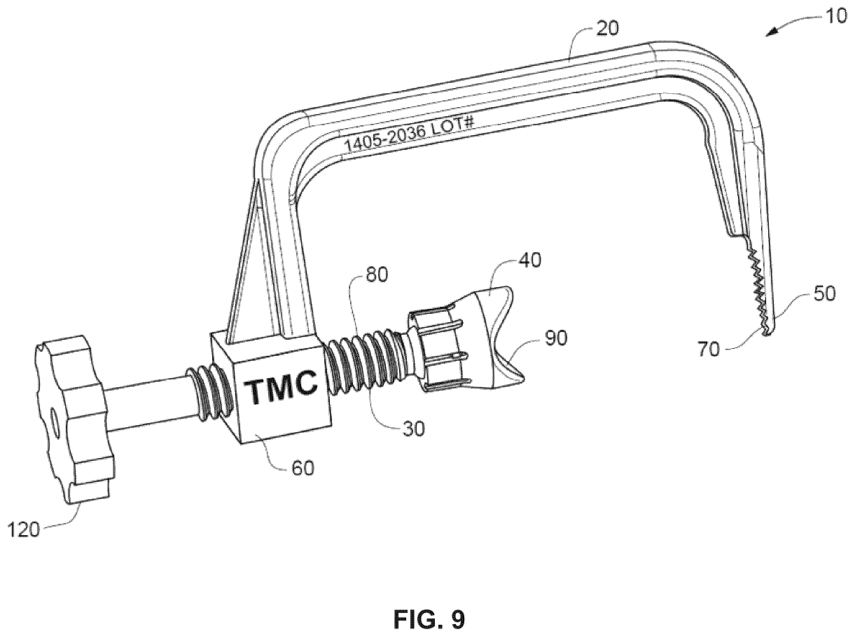

FIG. 9 is a perspective illustration of an example bone positioning device 10 that can be used to move first metatarsal 210. Additional details on bone positioning device 10 can be found in U.S. Pat. No. 9,936,994, titled “BONE POSITIONING GUIDE,” and issued Apr. 10, 2018, the entire contents of which are incorporated herein by reference.

In the example of FIG. 9, a bone positioning device 10 (which may also be referred to as a bone positioning guide) includes a main body member 20, a shaft 30, a bone engagement member 40 connected to the shaft, and a tip 50 is connected to the main body member. In general, the main body member 20 can be sized and shaped to clear anatomy or other instrumentation (e.g., pins and guides) while positioned on a patient. As illustrated, the main body member 20 includes a generally C-shaped configuration with a first end 60 and a second end 70. In some embodiments, the main body is sized and configured to engage a metatarsal to be moved with bone engagement member 40 and another metatarsal that functions as an anchoring bone with tip 50. Although bone positioning device 10 is illustrated as being formed of two components, main body member 20 and shaft 30, the guide can be fabricated from more components (e.g., three, four, or more) that are joined together to form the guide.

Shaft 30 can be movably connected to the main body member 20 proximate its first end 60. In some embodiments, the shaft 30 includes threads 80 that engage with the main body member 20 such that rotation of the shaft translates the shaft with respect to the main body member. In other embodiments, the shaft can slide within the main body member and can be secured thereto at a desired location with a set screw. In yet other embodiments, the shaft can be moved with respect to the main body by a ratchet mechanism, e.g., with or without a third device or by the clinician's hand pressure.

To move the ends of bone positioning device 10 relative to each other to move a bone engaged therewith, bone positioning device 10 can also include an actuator (e.g., a knob or a handle) 120 to actuate the mechanism. In the embodiment shown, the actuator can be useful for allowing a user to rotate the shaft with respect to the main body member 20. As shown, the actuator, shaft, and bone engagement member may include a cannulation to allow the placement of a fixation wire (e.g., K-wire) through these components and into contact with or through a bone engaged with the bone engagement member. For example, the fixation wire can be placed into the bone engaged with bone engagement member 40 to fix the position of the bone engagement member with respect to the bone. In another example, the fixation wire can be placed through the bone in contact with the bone engagement member and into an adjacent bone to maintain a bone position of the bone in contact with the bone engagement member and the adjacent bone.

While FIG. 9 illustrates one example configuration of a bone positioning device, the disclosure is not limited to the use of a bone positioning device with such an illustrated configuration. As one alternative example, instead of being configured to engage a metatarsal to be moved with bone engagement member 40 and another metatarsal with tip 50, the bone positioning device may be configured to extend from a metatarsal to be moved to a bone other than a metatarsal, such as a cuneiform to which the bone positioning device is anchored. As another example, a bone positioning device may utilize a tenaculum or tong structure to move one end engaged with the metatarsal relative to another anchoring bone. As a further example, a band, suture, and/or other interconnecting member may be attached to the metatarsal and a bone other than the metatarsal and then used to apply and/or translate a force to move the metatarsal.

In yet further examples, the clinician may use an instrument to apply a force to move the metatarsal without having the instrument simultaneously contact the metatarsal and a bone other than the metatarsal, e.g., in addition to or in lieu of using an bone positioning device that contacts both the metatarsal being moved and a bone other than the metatarsal. For example, the clinician may insert a pin into the metatarsal and manually manipulate the pin to control movement of the metatarsal. As another example, the clinician may insert one pin into a metatarsal and another pin into a bone other than the metatarsal (e.g., one into first metatarsal 210 and one into second metatarsal 212). The clinician may then squeeze the two pins together.

Independent of the configuration of the bone positioning device used (in instances where a bone positioning device is used), the bone positioning device may be configured to move the metatarsal being realigned in at least one plane (e.g., relative to the opposed proximal phalanx) and/or an adjacent metatarsal, such as at least two planes, or all three planes. The at least one plane can be the transverse plane, the frontal plane, and/or the sagittal plane. In some embodiments, actuation of the bone positioning device moves the metatarsal in at least the transverse plane to close the intermetatarsal angle (“IMA”), e.g., between first metatarsal 210 and second metatarsal 212. Actuation of the bone positioning device may reduce the IMA to an angle less than 12 degrees, such as less than 10 degrees, less than 8 degrees, less than 6 degrees, or less than 4 degrees.

In addition to or in lieu of moving the metatarsal in the transverse plane, actuation of the bone positioning device may realign the metatarsal in the frontal plane. A normal first metatarsal will be positioned such that its crista prominence is generally perpendicular to the ground and/or its sesamoid bones are generally parallel to the ground and positioned under the metatarsal. This position can be defined as a metatarsal rotation of 0 degrees. In a misaligned first metatarsal, the metatarsal may be axially rotated between about 4 degrees to about 30 degrees or more. In some embodiments, the metatarsal is moved in the frontal plane to reducing the metatarsal rotation from about 4 degrees or more to less than 4 degrees (e.g., to about 0 to 2 degrees) by rotating the metatarsal.

In some implementations, such as when utilizing a bone positioning device 10 as illustrated in FIG. 9, actuation of the bone positioning device may simultaneously move the metatarsal in two planes to establish a moved position with respect to those planes. For example, actuating bone positioning device 10 may cause first metatarsal 210 to move in the transverse plane to close the IMA and in the frontal plane to move the crista prominence toward a 0 degree rotation angle.

When actuating the bone positioning device, the metatarsal may or may not also move in the sagittal plane (e.g., plantarly). For instance, in some implementations, actuating the bone positioning device causes the metatarsal to move in the transverse plane to close the IMA and the frontal plane to reduce the degree of rotation without any significant movement in the sagittal plane (e.g., such that there is less than 5 degrees of sagittal plane movement). This can maintain the sagittal plane declination of the first metatarsal while the position of the metatarsal is corrected in two other planes.

In some such example, the clinician may or may not perform a separate step to adjust the position of the metatarsal in the sagittal plane. For example, either before or after adjusting the position of the metatarsal in the transverse plane and/or frontal plane, the clinician may adjust the position of the metatarsal in the sagittal plane to help set a desired amount of dorsiflexion. For example, after moving first metatarsal 210 in the transverse plane and the frontal plane using bone positioning device 10, the clinician may manually grasp the metatarsal (e.g., by grasping the metatarsal directly, grasping a pin connected to the metatarsal, and/or with the use of a tong-type instrument) and move the first metatarsal in the sagittal plane. Bone positioning device 10 may continue to engage the metatarsal while the metatarsal is being moved in the sagittal plane, e.g., the help hold the moved position of the metatarsal in the frontal and/or transverse planes.

In some examples, the clinician moves first metatarsal 210 in the sagittal plane to help a declination angle (plantarflexion) for the first metatarsal that is substantially the same as the declination angle exhibited by an adjacent metatarsal (e.g., second metatarsal). The declination angle of the first metatarsal relative to the second metatarsal may be observed by taking a lateral X-ray image of foot 200. In some examples, the clinician moves first metatarsal 210 in the sagittal plane to set a declination angle relative to ground that is within approximately 5 degrees (plus or minus) of the declination angle of the second metatarsal, such as less than 3 degrees (plus or minus) different than the declination angle of the second metatarsal, or less than 2 degrees (plus or minus) different than the declination angle of the second metatarsal. In addition to or in lieu of moving the first metatarsal 210 relative to a declination angle of the second metatarsal 212, the clinician may move the metatarsal in the sagittal plane to establish a declination angle relative to ground, such as an angle ranging from 10 to 35 degrees with respect to ground, or from 20 to 25 degrees with respect to ground.

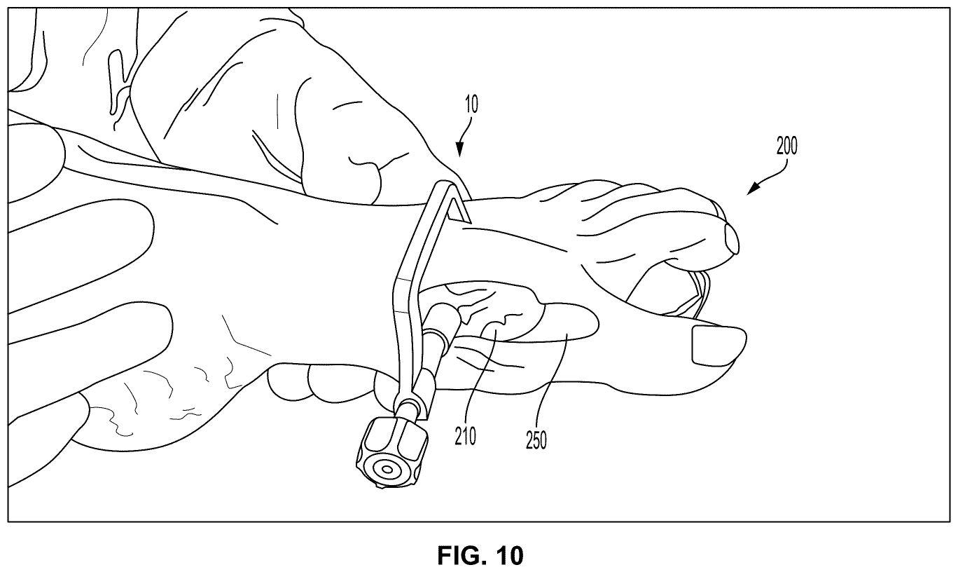

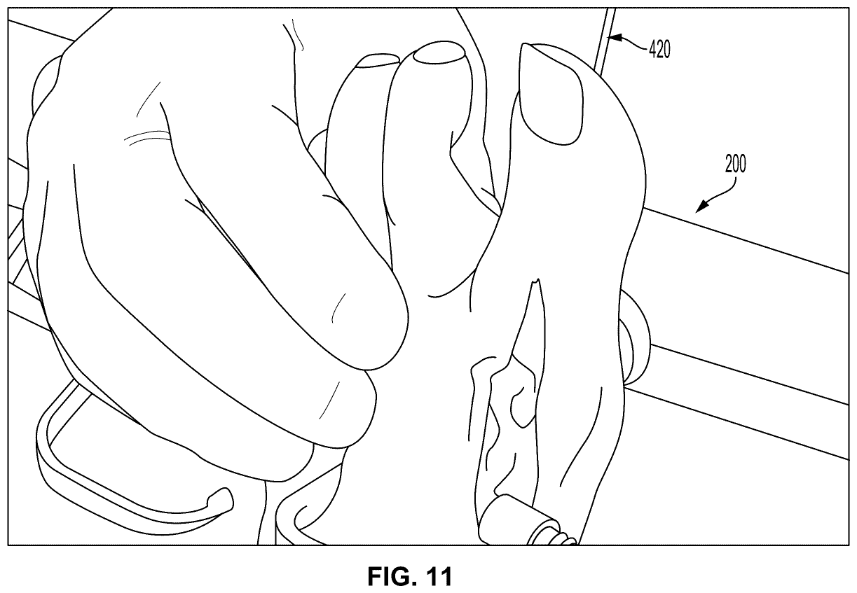

FIG. 10 is a perspective view of foot 200 showing bone positioning device 10 attached to the foot. In the example, the bone engagement member of bone positioning device 10 is placed in contact with first metatarsal 210, such as on the medial ridge of the first metatarsal. The tip 50 of bone positioning device 10 is placed through a stab incision on the lateral cortex of the second metatarsal 212. Actuation of bone positioning device 10 can cause a first ray deformity to be reduced on in the transverse and frontal planes.

If desired, a clinician may utilize a fulcrum between the metatarsal being moved and an adjacent metatarsal, e.g., to prevent compression of the metatarsals together during actuation of the bone positioning device. When used, the fulcrum may be inserted in the intermetatarsal space, e.g., between the distal head of the first metatarsal 210 and the adjacent second metatarsal 212. Additionally or alternatively, the fulcrum may be inserted between the proximal base of the metatarsal and the proximal base of an adjacent metatarsal (e.g., between the proximal base of the first and second metatarsals 210, 212), such as be making a stab incision at the base between the two metatarsals and inserting the fulcrum through the incision. Details on example fulcrum structures that may be used can be found in U.S. Pat. No. 10,342,590, issued Jul. 9, 2019 and titled “TARSAL-METATARSAL PROCEDURE UTILZING FULCRUM,” the entire contents of which are incorporated herein by reference.

While the foregoing description has described the metatarsal as being moved in at least one plane with the aid of a bone positioning device, in other examples, the clinician may move the metatarsal by hand without the aid of a bone positioning device providing controlled movement of the metatarsal relative to another bone that the bone positioning device is also engaged with. In these alternative examples, the clinician may generally move the metatarsal in at least one plane, such as at least two planes (e.g., any two of the frontal, transverse, and sagittal planes), or all three planes. The clinician can move the metatarsal in one plane at a time, resulting in multiple different movements to achieve the multi-planar movement. Additionally or alternatively, the clinician may perform a single repositioning of the metatarsal in multiple planes. The clinician may move the metatarsal to any of the positions and/or angular ranges discussed above with respect to movement achieved when also using a bone positioning device.

Independent of whether the clinician utilizes a bone positioning device or moves the metatarsal without the aid of bone positioning device, the clinician may or may not insert two parallel reference pins to help visualize the bone movement. For example, prior to moving the metatarsal 210, the clinician may insert a first pin into the metatarsal and a second, parallel pin into the first proximal phalanx 250. The two pins may project out of the respective bones. As the bones are moved in three-dimensional space and/or relative to each other, the orientation of the two pins may shift out of parallel alignment. This may provide a visual guide to the clinician on the extent of relative movement between the bones.

In the example technique of FIG. 4, the first proximal phalanx 250 opposing the metatarsal 210 may be moved in at least one plane in addition to or in lieu of moving the metatarsal (310). For example, either before or after moving the first metatarsal 210 in at least one plane, the proximal phalanx 250 may be moved in at least one plane (e.g., any one of the frontal, transverse, and sagittal planes), such as at least two planes. For example, after moving the first metatarsal 210 in one, two, or three planes, the clinician may move the proximal phalanx in the sagittal plane and frontal plane relative to the moved metatarsal.

To help facilitate positioning of the proximal phalanx 250 relative to the first metatarsal 210, the clinician may provisionally fixate the position of the moved metatarsal. As one example, the clinician may insert a pin (e.g., a guide wire or K-wire) through the moved metatarsal and into another bone, such as an adjacent metatarsal (e.g., second metatarsal 212). When using bone positioning device 10, the pin can be inserted through the cannula in the actuator, through the first metatarsal, and into the second metatarsal. Alternatively, the bone positioning device used to apply a force to move the metatarsal may remain engaged with the bone (e.g., with actuator optionally locked), thereby functioning to hold the metatarsal in the moved position for subsequent realignment of the proximal phalanx 250. In still other examples, the moved metatarsal may not be provisionally held with a pin and/or instrumentation while adjusting the alignment of the proximal phalanx 250.

In one implementation, the clinician moves the proximal phalanx 250 and sets the position of the proximal phalanx using a pin extending generally parallel to the long axis of the proximal phalanx. For example, after moving the first metatarsal 210 to a desired position, the clinician may insert a pin (e.g., guide wire, K-wire) from the distal end of the hallux toward the moved metatarsal. The clinician can advance the pin in a distal to proximal direction through the distal phalanx 260 followed by the proximal phalanx 250 and into the end face of the first metatarsal 210. Prior to advancing the pin out of the proximal end face of the proximal phalanx 250 and into the distal end face of the first metatarsal 210, the clinician may move the proximal phalanx 250 (and attached distal phalanx 260) to a desired sagittal plane position and/or transverse plane position and/or frontal plane position. With the proximal phalanx 250 moved to a desired position in the sagittal plane, the clinician may advance the pin out of the proximal end face of the proximal phalanx 250 and into the distal end face of the first metatarsal 210, thereby provisionally fixating the proximal phalanx 250 to the first metatarsal 210 and holding the set sagittal plane position for the proximal phalanx. Additional details on example pin insertion and placement techniques may be found in U.S. Pat. No. 9,622,805, titled “BONE POSITIONING AND PREPARING GUIDE SYSTEMS AND METHODS,” issued Apr. 18, 2017, the entire contents of which are incorporated herein by reference.

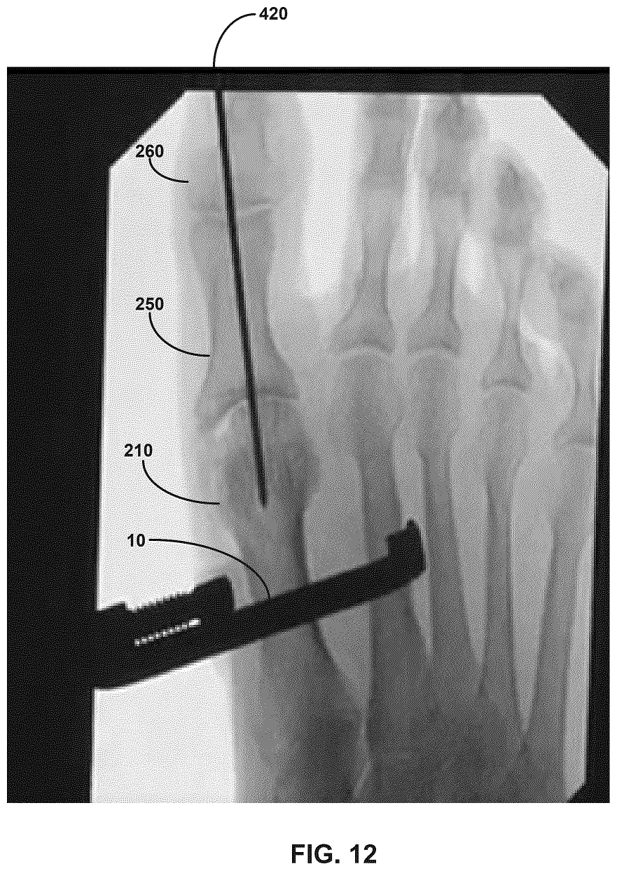

FIG. 11 is a perspective view of foot 200 showing an example pin 420 inserted into the hallux of the foot and into the first metatarsal, e.g., to provisionally fixate the proximal phalanx relative to the metatarsal at a desired angular position in the sagittal plane. Pin 420 is shown projecting out the distal end of the hallux for visualization by the clinician. FIG. 12 is an X-ray of the foot 200 of FIG. 11 taken in the dorsal to plantar direction. The X-ray shows how pin 420 is inserted through distal phalanx 260, proximal phalanx 250, and into first metatarsal 210.

A variety of alternative provisional fixation instruments and/or techniques can be used in addition to or in lieu of a pin inserted proximally through the proximal phalanx 250 and into the first metatarsal 210. For example, a pin may be insert through the first metatarsal 210 into the proximal phalanx (e.g., from the dorsal side of the first metatarsal and advanced distally and plantarly across the MTP joint). As another example, a pin may be inserted into the proximal end of the proximal phalanx 250 to a depth sufficient to provide a small section of pin protruding out of the proximal end of the proximal phalanx. The pin may be positioned with distal end of the pin protruding out another side of the proximal phalanx. In either case, the proximal phalanx 250 can then be moved to a desired position with respect to the metatarsal 210. The pin can then be driven back through the proximal phalanx 250, e.g., by engaging the distal end of the pin with a driver mechanism, and into another bone, such as the opposed metatarsal 210.

In some examples, the clinician moves proximal phalanx 250 in the sagittal plane to help set a dorsiflexion angle measured relative to ground ranging from 0 degrees to 20degrees, such as from approximately 5 degrees to approximately 10 degrees of dorsiflexion relative to the ground. The clinician may place the foot (or at least the metatarsal and hallux portion of the foot) on a planar surface, such a planar surface of a board or block. The clinician may then visualize and set the desired angle of proximal phalanx 250 in the sagittal plane relative to the planar surface. For example, the clinician may position the weight bearing surface of the foot in contact with the planar surface and view the position of the hallux interphalangeal joint, e.g., as pin 420 is being inserted and/or the position of the proximal phalanx is being adjusted in the sagittal plane. The clinician may set the plantar head of the proximal phalanx 250 at an angle of inclination relative where the weight bearing surface contacts the planar surface, e.g., within any of the foregoing angular ranges discussed above. In some examples, the clinician sets the plantar head of the proximal phalanx 250 to be positioned a distance less than 4 mm off of the planar surface contacted by the weight bearing surface of the foot, such as less than 3 mm, or a distance ranging from approximately 1 mm to approximately 2 mm.

In addition to or in lieu of positioning proximal phalanx 250 in the sagittal plane, the clinician be move the proximal phalanx in the transverse plane. The clinician may move the proximal phalanx to be substantially parallel to an adjacent proximal phalanx, e.g., such as by moving the first proximal phalanx 250 to be parallel to the second proximal phalanx 252.

With proximal phalanx 250 optionally provisionally fixated to first metatarsal 210 using pin 420, the clinician may move the proximal phalanx in the front plane to set a desired frontal plane position of the proximal phalanx. For example, the clinician can utilize pin 420 as a frontal plane axis of rotation and rotate proximal phalanx 250 about the pin to a desired frontal plane rotation position. The clinician may use the hallux and/or toenail as a reference for neutral or slight supination alignment of the hallux. The clinician may de-rotate proximal phalanx 250 until there is substantially no frontal plane rotation of the hallux.

With the proximal phalanx 250 moved to a desired position, the clinician may utilize intra-operative fluoroscopy to confirm the position of the first metatarsal 210 and/or proximal phalanx 250. For example, the clinician may generate fluoroscopic images in both the anterior-posterior view and lateral view to visualize the position of the bones in three-dimensional space. If needed, corrective repositioning of the metatarsal and/or proximal phalanx can be performed. Otherwise, if the clinician is satisfied with the positioning of the bones, the clinician may permanently fixate the MTP joint for fusion with the metatarsal and proximal phalanx in their relative moved positions.

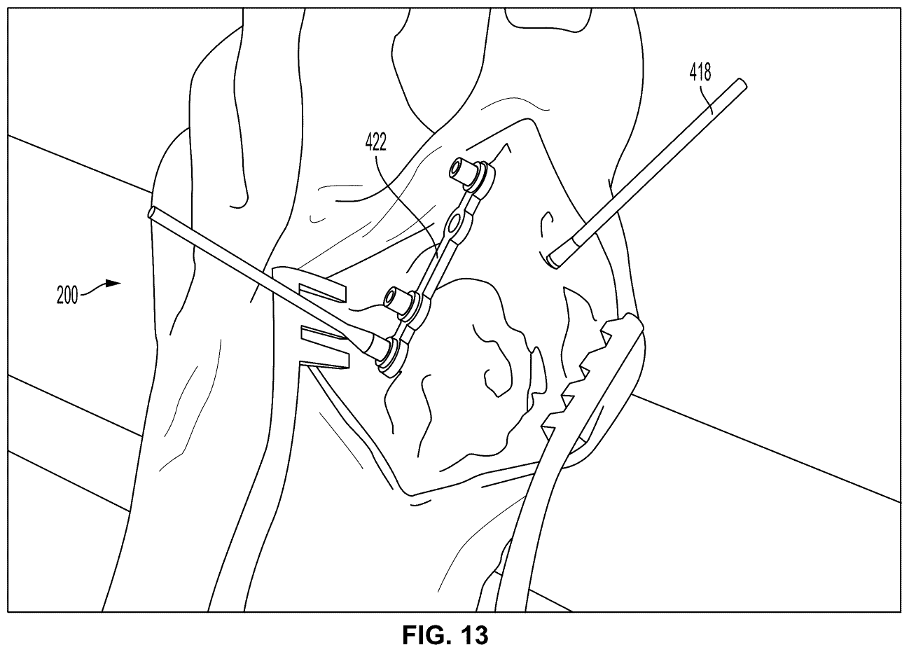

In some examples, the clinician may compress the prepared end face of the proximal phalanx against the prepared end face of the metatarsal to facilitate fusion together prior to applying one or more permeant fixation devices. For example, the clinician use pin 420 to compress the proximal phalanx 250 to the first metatarsal 210, compressing the MTP joint. Additionally or alternatively, the clinician may insert a compression pin (e.g., a screw, a partially threaded olive wire, a K-wire, etc.) to compress across the MTP joint, e.g., by inserting the compression member from the dorsal-medial base of the proximal phalanx 250 across the first MTP joint 232 into the proximal-lateral first metatarsal shaft 210 for compression. When pin 420 is already inserted into the bones, the second pin may stabilize the frontal-plane correction of the phalanx. FIG. 13 is a perspective view of foot 200 showing a compression pin 418 inserted through the proximal phalanx and into the first metatarsal along with an example bone plate 422 being applied across the MTP joint.

To compress the prepared end of proximal phalanx 250 against the prepared end of first metatarsal 210 for fixation and fusion, a compressor instrument may optionally be used.

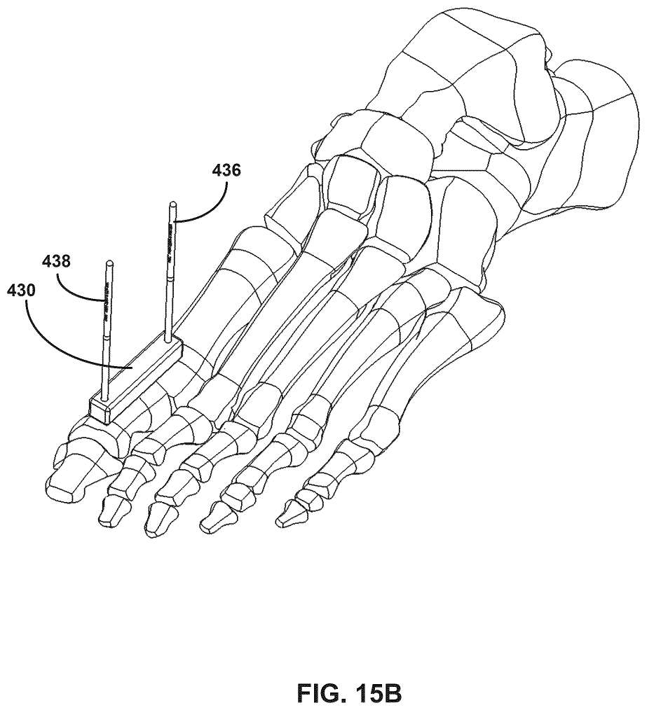

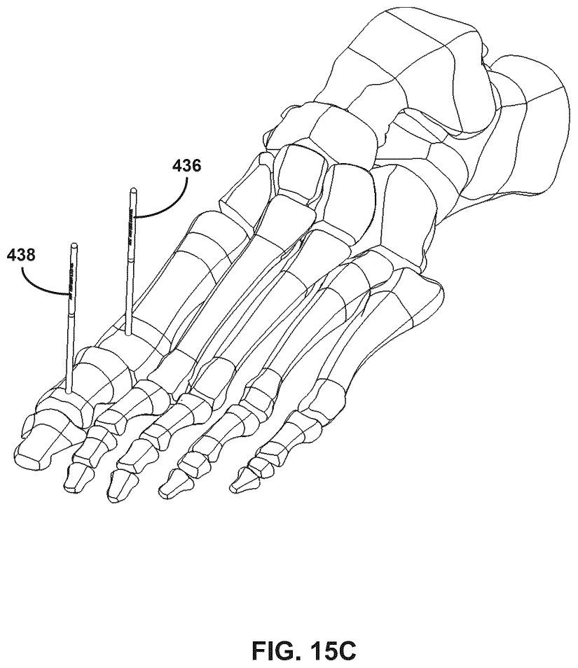

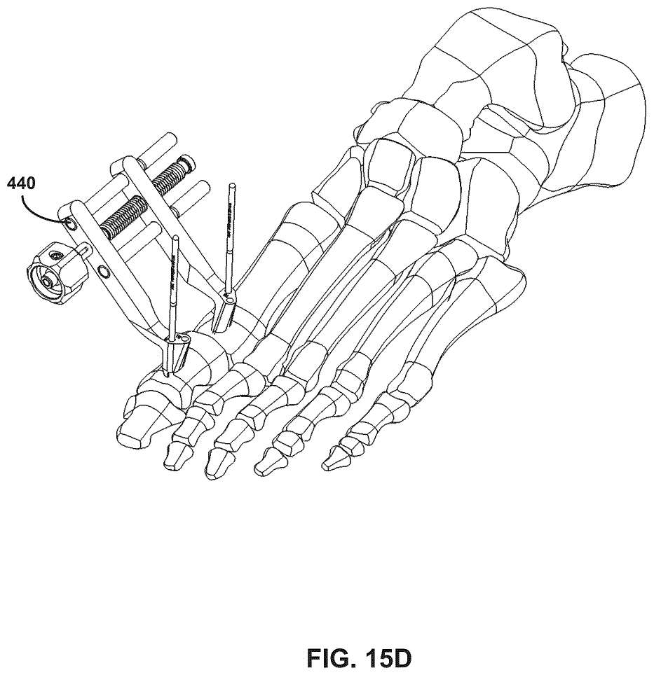

FIGS. 15A-15D are illustrations of example surgical steps that may be performed to engage a compressor with a metatarsal and opposed proximal phalanx to facilitate compression. Some or all of the example steps of FIGS. 15A-15D up to compression may be performed before or after the end faces of the metatarsal and/or proximal phalanx are prepared and/or before or after realignment of the metatarsal and proximal phalanx. Further, while the example steps of FIGS. 15A-15D are described in connection with engaging a compressor device, the steps of FIGS. 15A-15C may additionally or alternatively be utilized to insert generally parallel guide pins into the metatarsal and opposed proximal phalanx, e.g., to help visualize realignment of one or both bone portions.

With reference to FIG. 15A, an optional guide 430 may be positioned on the first metatarsal 210 and/or opposed proximal phalanx 250. Guide 430 can define one or more apertures through which one or more corresponding pins can be insert. For example, guide 430 may cross the MTP joint and define at least one aperture 432 positioned over the first metatarsal 210 for receiving a pin and at least one aperture 434 positioned over the proximal phalanx for receiving a pin. The apertures may be oriented relative to each other to place the pins inserted therethrough at any desired orientation with respect to each other, such as generally parallel to each other and/or biased plantarly.

FIG. 15B illustrates a first pin 436 inserted through aperture 432 into first metatarsal 210 and a second pin 438 inserted through aperture 434 into proximal phalanx 250. In this example, the first and second pins 436, 438 extend dorsally and are generally parallel to each other. With the pin(s) positioned using optional guide 430, the guide may be removed as illustrated in FIG. 15C.

After suitably preparing and moving the first metatarsal 210 and proximal phalanx 250, the end face of the proximal phalanx can be compressed against the end face of the metatarsal. In some examples, an axial wire or pin may be placed to extend proximally out of the end face of the proximal phalanx. When used, the proximal phalanx may slide along the wire or pin during compression. In either case, a compressor instrument 440 may be installed on the first pin 436 and the second pin 438 and then engaged to compress the bones attached to the pins together, as illustrated in FIG. 15D. Additional details on compressor instrumentation that may be used can be found in U.S. Patent Publication 2020/0015856, filed Jul. 11, 2019 and titled “COMPRESSOR-DISTRACTOR FOR ANGULARLY REALIGNING BONE PORTIONS,” the entire contents of which are incorporated herein by reference.

With reference to FIG. 4, the example technique includes applying at least one bone fixation device across or through the MTP joint separating the metatarsal from the opposed proximal phalanx (312). Any one or more bone fixation devices can be used including, but not limited to, a compressing bone screw, a bone plate, a bone staple, an external fixator, and/or an intramedullary implant. The bone fixation device may be secured on one side to the metatarsal, bridge the MTP joint, and be secured on an opposite side to the proximal phalanx.

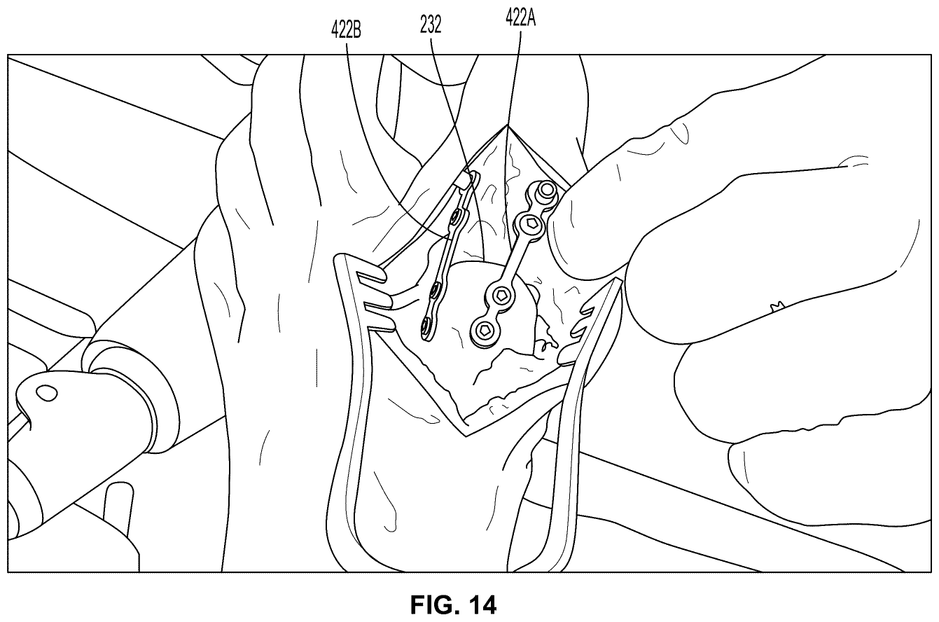

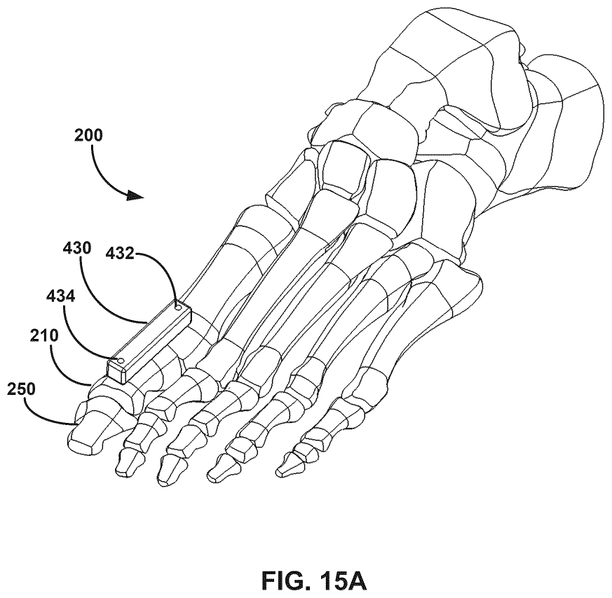

In one example, two bone plates may be placed across the MTP joint to provide bi-planar plating. For example, a first bone plate may be positioned on a dorsal-medial side of the metatarsal and proximal phalanx. A second bone plate may be positioned on a medial-plantar side of the metatarsal and the proximal phalanx. Independent of the number or configuration of bone plates, the plates may be applied with the insertion of bone screws. FIG. 14 is a perspective view of foot 200 showing example bi-planar bone plates 422A, 422B applied across the MTP joint to allow the metatarsal to fuse to the proximal phalanx during subsequent recovery.

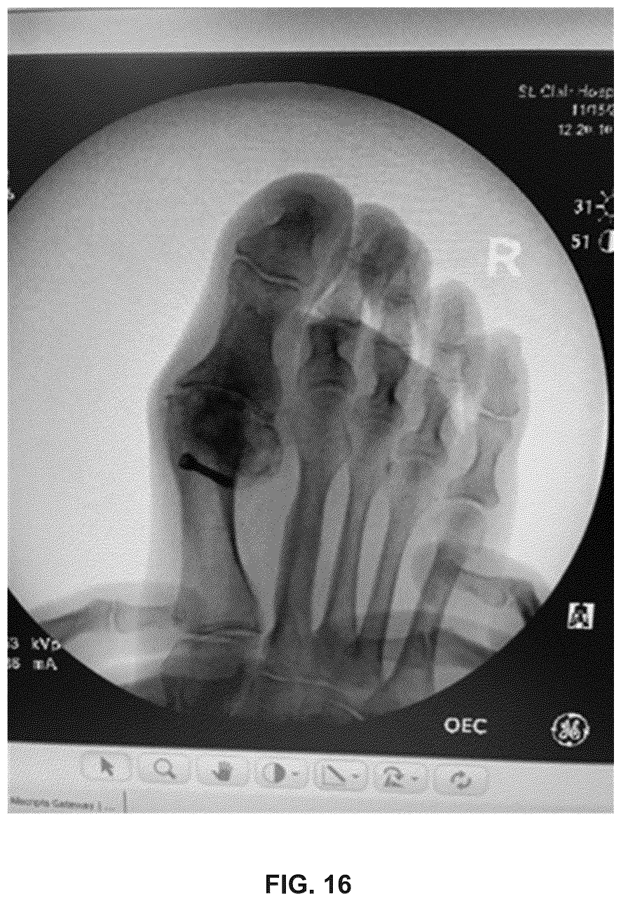

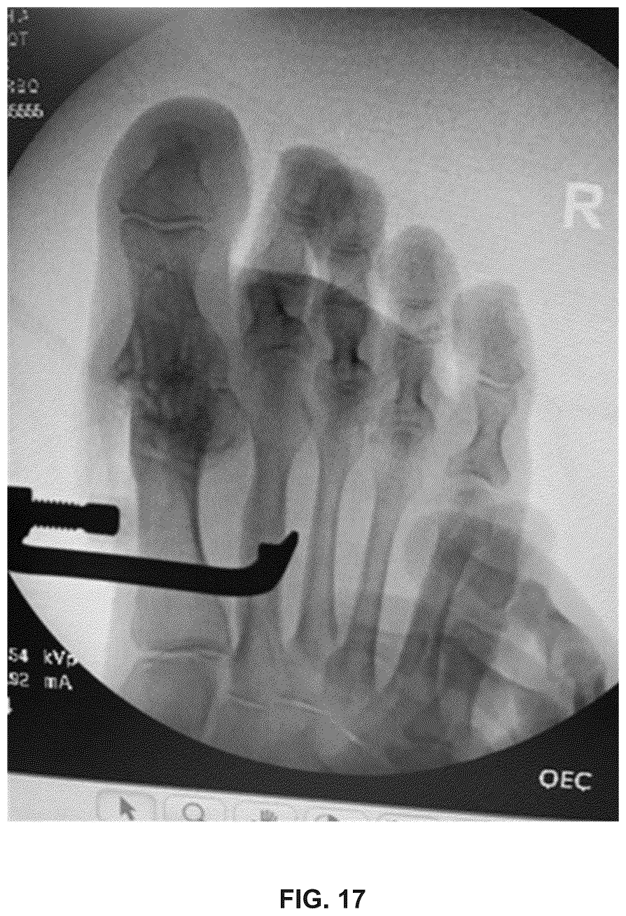

FIGS. 16 and 17 are pre-and post-operative fluoroscopic images of a foot showing example bone realignment according to the techniques of the present disclosure.

Various examples have been described. These and other examples are within the scope of the following claims.

1. A method of preparing a metatarsophalangeal joint for fusion, the method comprising:
surgically accessing a metatarsophalangeal joint separating a metatarsal from an opposed proximal phalanx;
preparing an end of the metatarsal and preparing an end of the opposed proximal phalanx;
moving the metatarsal in at least two planes to establish a moved position of the metatarsal;
applying at least one bone fixation device at least one of across and through the metatarsophalangeal joint separating the metatarsal from the opposed proximal phalanx.

2. The method of claim 1, wherein moving the metatarsal in at least two planes comprises moving the metatarsal in three planes.

3. The method of claim 2, wherein moving the metatarsal comprises:
moving the metatarsal in a frontal plane and a transverse plane, and
after moving the metatarsal in the frontal plane and the transverse plane, moving the metatarsal in a sagittal plane.

4. The method of claim 1, wherein moving the metatarsal in at least two planes comprises reducing an angle between the metatarsal and an adjacent metatarsal to less than 12 degrees.

5. The method of claim 1, wherein moving the metatarsal comprises moving the metatarsal after preparing the end of the metatarsal and after preparing the end of the opposed phalanx.

6. The method of claim 1, further comprising moving the opposed proximal phalanx relative to the metatarsal in at least a sagittal plane.

7. The method of claim 6, further comprising moving the opposed proximal phalanx in a frontal plane.

8. The method of claim 7, further comprising inserting a pin through the opposed proximal phalanx and into the metatarsal, wherein moving the opposed proximal phalanx in the frontal plane comprises rotating the proximal phalanx about the pin.

9. The method of claim 1, further comprising repositioning a medial sesamoid bone associated with the metatarsal and a lateral sesamoid bone associated with the metatarsal on opposite sides of a sagittal plane.

10. The method of claim 9, wherein repositioning the medial sesamoid bone and the lateral sesamoid bone comprises releasing the medial sesamoid bone and the lateral sesamoid bone with an instrument and repositioning the medial sesamoid bone and the lateral sesamoid bone prior to moving the metatarsal in the at least two planes.

11. The method of claim 1, further comprising engaging a bone positioning guide with the metatarsal and a bone other than the metatarsal, wherein moving the metatarsal in the at least two planes comprises actuating the bone positioning guide and thereby moving the metatarsal in at least one of the two planes using the bone positioning guide.

12. The method of claim 11, wherein actuating the bone positioning guide comprises actuating the bone positioning guide and thereby moving the metatarsal in at least a frontal plane and a transverse plane.

13. The method of claim 11, wherein engaging the bone positioning guide with the metatarsal and the bone other than the metatarsal comprises engaging the bone positioning guide with the metatarsal and another metatarsal.

14. The method of claim 11, wherein the bone positioning guide comprises:
a first end configured to engage the metatarsal and a second end configured to engage the bone other than the metatarsal, and
a mechanism configured to move the first end and the second end toward each other.

15. The method of claim 14, wherein the mechanism comprises at least one of a ratchet and a threaded shaft.

16. The method of claim 1, wherein preparing the end of the metatarsal and preparing the end of the opposed proximal phalanx each comprises removing cartilage down to subchondral bone.

17. The method of claim 1, wherein:
preparing the end of the metatarsal comprises reaming the end of the metatarsal with a generally conical-shaped reamer, and
preparing the end of the opposed proximal phalanx comprises reaming the end of the opposed phalanx with a generally cone-shaped reamer.

18. The method of claim 1, further comprising, prior to applying the at least one bone fixation device, provisionally fixing the moved position of the metatarsal.

19. The method of claim 18, wherein provisionally fixing the moved position of the metatarsal comprises inserting a pin into the metatarsal and a bone other than the metatarsal.

20. A method of preparing a metatarsophalangeal joint for fusion, the method comprising:
surgically accessing a metatarsophalangeal joint separating a metatarsal from an opposed proximal phalanx;
preparing an end of the metatarsal and preparing an end of the opposed proximal phalanx;
engaging a bone positioning guide with the metatarsal and a bone other than the metatarsal;
actuating the bone positioning guide and thereby moving the metatarsal in at least one plane to establish a moved position of the metatarsal;
applying at least one bone fixation device at least one of across and through the metatarsophalangeal joint separating a metatarsal from an opposed proximal phalanx.
